(19) United States
(12) Patent Application Publication
(10) Pub. No.: US 2017/0168000 A1
(43) Pub. Date: Jun. 15, 2017

(54) GAS SENSOR AND ORGANIC TRANSISTOR

(71) Applicant: FUJIFILM CORPORATION, Tokyo (JP)

(72) Inventor: Takahiko ICHIKI, Kanagawa (JP)

(73) Assignee: FUJIFILM CORPORATION, Tokyo (JP)

(21) Appl. No.: 15/442,775

(22) Filed: Feb. 27, 2017

Related U.S. Application Data
(63) Continuation of application No. PCT/JP2015/073718, filed on Aug. 24, 2015.

(30) Foreign Application Priority Data
Sep. 24, 2014 (JP) 2014-193770

(51) Int. Cl. G01N 27/12; H01L 51/05; G01N 33/00

(52) U.S. Cl. CPC G01N 27/125; G01N 33/0027; H01L 51/0558; H01L 51/0545

(57) ABSTRACT

The present invention provides a gas sensor which exhibits high detection sensitivity and includes an organic transistor and an organic transistor. A gas sensor of the present invention includes a bottom-gate type organic transistor including a source electrode, a drain electrode, a gate electrode, a gate insulating layer, an organic semiconductor layer, and a receptor layer which is disposed between the gate insulating layer and the organic semiconductor layer and includes a compound that interacts with gas molecules which are a detection subject.

### CROSS-REFERENCE TO RELATED APPLICATIONS

This application is a Continuation of PCT International Application No. PCT/JP2015/073718 filed on Aug. 24, 2015, which claims priority under 35 U.S.C. §119(a) to Japanese Patent Application No. 2014-193770 filed on Sep. 24, 2014. The above application is hereby expressly incorporated by reference, in its entirety, into the present application.

### BACKGROUND OF THE INVENTION

1. Field of the Invention

The present invention relates to a gas sensor and an organic transistor.

2. Description of the Related Art

As field-effect transistors (FET), RF identification tags (RFID), and the like which are used in liquid crystal displays or organic electroluminescent (EL) displays, organic transistors (organic TFT) are used since it is possible to reduce weight and costs and impart flexibility.

Organic transistors are applied to a variety of applications, and, for example, in JP2006-258661A, organic transistors are applied to biosensors. More specifically, JP2006-258661A discloses organic transistors in which a surface layer responding to target molecules is disposed on the surface of an organic semiconductor layer.

### SUMMARY OF THE INVENTION

Meanwhile, in recent years, there has been a demand for the development of gas sensors capable of detecting gas molecules with higher sensitivity.

The present inventors produced an organic transistor in which a receptor layer including a compound that interacts with gas molecules is disposed on the outermost side with reference to the constitution of FIG. 1 in JP2006-258661A and studied the performance of a gas sensor produced using the organic transistor. As a result, it was found that the sensitivity of the gas sensor did not always reach the level required at the moment and additional improvements are required.

In addition, when a receptor layer is produced immediately on an organic semiconductor layer as in the constitution of JP2006-258661A, there are cases in which the organic semiconductor layer is damaged, and consequently, there is a concern that the performance of organic transistors may degrade.

The present invention has been made in consideration of the above-described circumstance, and an object of the present invention is to provide a gas sensor which exhibits high detection sensitivity and includes an organic transistor.

In addition, another object of the present invention is to provide an organic transistor that is used for the gas sensor.

The present inventors carried out intensive studies regarding the above-described objects and consequently found that desired effects can be obtained by controlling the location of a receptor layer and completed the present invention. That is, the present inventors found that the above-described objects can be achieved by the following constitutions.

(1) A gas sensor comprising: a bottom-gate type organic transistor including a source electrode, a drain electrode, a gate electrode, a gate insulating layer, an organic semiconductor layer, and a receptor layer which is disposed between the gate insulating layer and the organic semiconductor layer and includes a compound that interacts with gas molecules which are a detection subject.

(2) The gas sensor according to (1), in which the organic transistor includes the gate electrode, the gate insulating layer disposed on the gate electrode, the receptor layer disposed on the gate insulating layer, the organic semiconductor layer disposed on the receptor layer, and the source electrode and the drain electrode disposed on the organic semiconductor layer.

(3) The gas sensor according to (1), in which the organic transistor includes the gate electrode, the gate insulating layer disposed on the gate electrode so as to cover the gate electrode, the source electrode and the drain electrode disposed on the gate insulating layer, the receptor layer that covers a surface of the gate insulating layer between the source electrode and the drain electrode, and the organic semiconductor layer disposed on the receptor layer.

(4) The gas sensor according to any one of (1) to (3), in which the organic semiconductor layer is a polycrystalline layer.

(5) The gas sensor according to any one of (1) to (4), in which the compound that interacts with gas molecules which are a detection subject has an amino group.

(6) The gas sensor according to any one of (1) to (5), in which a thickness of the receptor layer is 10 to 50 nm.

(7) The gas sensor according to any one of (1) to (6), in which a thickness of the organic semiconductor layer is 50 nm or less.

(8) The gas sensor according to any one of (1) to (7), in which the gas molecules which are a detection subject are gas molecules included in exhaled air of human beings.

(9) The gas sensor according to any one of (1) to (8), in which the gas molecules which are a detection subject are acetone.

(10) The gas sensor according to any one of (1) to (8), in which the gas molecules which are a detection subject are ethanol.

(11) A bottom-gate type organic transistor for a gas sensor, comprising: a source electrode, a drain electrode, a gate electrode, a gate insulating layer, an organic semiconductor layer, and a receptor layer which is disposed between the gate insulating layer and the organic semiconductor layer and includes a compound that interacts with gas molecules which are a detection subject.

According to the present invention, it is possible to provide a gas sensor which exhibits high detection sensitivity and includes an organic transistor.

In addition, according to the present invention, it is also possible to provide an organic transistor that is used for the gas sensor.

### BRIEF DESCRIPTION OF THE DRAWINGS

FIG. 1 is a cross-sectional view of an organic transistor that is used in a first embodiment of a gas sensor of the present invention.

FIG. 2 is a cross-sectional view of an organic transistor that is used in a second embodiment of the gas sensor of the present invention.

### DESCRIPTION OF THE PREFERRED EMBODIMENTS

Hereinafter, a gas sensor of the present invention will be described.

Meanwhile, in the present specification, numerical ranges expressed using “to” include numerical values described before and after the “to” as the lower limit value and the upper limit value.

First, a characteristic of the present invention is that a receptor layer including a compound that interacts with predetermined gas molecules which are a detection subject is disposed between a gate insulating layer and an organic semiconductor layer. That is, the present inventors found that, when the adsorption of gas molecules is carried out at a location near a channel region between a source electrode and a drain electrode, the transistor characteristics (current characteristic change) are significantly affected and completed the present invention.

### First Embodiment

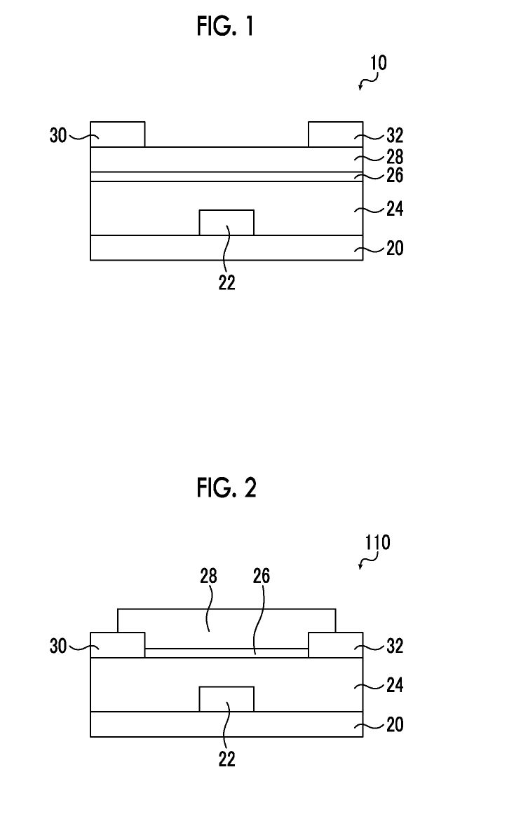

Hereinafter, a first embodiment of the gas sensor of the present invention will be described with reference to the drawing. FIG. 1 is a cross-sectional view of an organic transistor included in the first embodiment of the gas sensor. Meanwhile, the drawings in the present invention are schematic views, and the thickness relationship, location relationship, and the like between individual layers do not always match actual relationships, which shall apply to the following drawings.

The gas sensor includes an organic transistor that detects gas molecules of a detection subject and a measurement portion that detects the current characteristic change of the organic transistor and measures gas concentrations. As illustrated in FIG. 1, the organic transistor 10 included in the gas sensor includes a substrate 20, a gate electrode 22 disposed on the substrate 20, a gate insulating layer 24 disposed so as to cover the gate electrode 22, a receptor layer 26 disposed on the gate insulating layer 24, an organic semiconductor layer 28 disposed on the receptor layer 26, a source electrode 30 and a drain electrode 32 disposed to be spaced each other on the organic semiconductor layer 28. The organic transistor 10 is a so-called bottom-gate/top-contact type organic transistor.

In the gas sensor, gas molecules which are a detection subject in the receptor layer disposed in the organic transistor interact with a predetermined compound and are adsorbed to the receptor layer. Meanwhile, the gas molecules mainly pass through the organic semiconductor layer and reach the receptor layer. When the gas molecules are adsorbed in the receptor layer, the electrical resistance in the organic semiconductor layer disposed adjacent to the receptor layer changes, and consequently, the electrical characteristics of the organic transistor change (for example, the value of currents (drain currents) between the source electrode and the drain electrode changes). In the measurement portion connected to the organic transistor, the electrical characteristic changes of the organic transistor are detected, and gas concentrations are measured (computed) from the amounts of the changes. Meanwhile, although not illustrated, in the first embodiment of the gas sensor, the source electrode and the drain electrode in the organic transistor are electrically connected to the measurement portion.

Hereinafter, individual members constituting the gas sensor will be described in detail. First, the receptor layer 26 which is a characteristic of the present invention will be described in detail.

[Receptor Layer (Gas Molecule-Receiving Layer)]

The receptor layer 26 is a layer disposed between the gate insulating layer 24 and the organic semiconductor layer 28 and is a layer including a compound that interacts with predetermined gas molecules which are a detection subject. When the gas molecules are adsorbed to this layer, the electrical resistance of the organic semiconductor layer 28 changes, and consequently, the electrical characteristics of the organic transistor also change. The concentration of the gas molecules can be measured (computed) from the amount of the electrical characteristics changed.

The receptor layer 26 includes a compound that interacts with predetermined gas molecules which are a detection subject (hereinafter, also referred to as “gas-detecting compound”). The type of the gas-detecting compound is not particularly limited as long as the compound is capable of interacting with predetermined gas molecules which are a detection subject, and, for example, a compound having a group having at least one hetero atom selected from the group consisting of a nitrogen atom, an oxygen atom, and a sulfur atom is preferred. Compounds easily interact with gas molecules through the hetero atom as long as the compounds have the above-described group.

Among these, the gas-detecting compound preferably includes an amino group since the detection sensitivity of the gas sensor is superior. Meanwhile, conceptually, the scope of the amino group include a primary amino group (—NH₂), a secondary amino group (—NH—), and a tertiary amino group (>N—).

Meanwhile, the type of the interaction is not particularly limited, and examples thereof include the hydrogen bond, the electrostatic interaction, the Van der Waals action, and the like.

The gas-detecting compound may be a low-molecular-weight compound or a high-molecular-weight compound having a predetermined repeating unit. A high-molecular-weight compound is preferred from the viewpoint of the flatness of the receptor layer 26. Meanwhile, the low-molecular-weight compound is a compound that does not have a plurality of repeating units.

Specific examples of the gas-detecting compound include phorphyrin and derivatives thereof (for example, benzophorphyrin, tetraphenylphorphyrin, tetraphenylphorphyrin-manganese complexes), phthalocyanine or derivatives thereof, and the like. Meanwhile, in the phorphyrin and derivatives thereof and the phthalocyanine or derivatives thereof, a metal atom (for example, manganese, cobalt, iron, vanadium, molybdenum, ruthenium, or the like) may be included.

Alternatively, the gas-detecting compound may be a high-molecular-weight compound having the above-described compound in a side chain.

The content of the gas-detecting compound in the receptor layer 26 is not particularly limited, but is preferably 50% by mass or more, more preferably 80% by mass or more, and still more preferably 90% by mass or more of the total mass of the receptor layer 26 since the detection sensitivity of the gas sensor is superior. The upper limit is not particularly limited and is, for example, 100% by mass.

The thickness of the receptor layer 26 is not particularly limited, but is preferably 10 to 100 nm and more preferably 10 to 50 nm from the viewpoint of the balance between the thickness reduction of the gas sensor and the detection sensitivity of the gas sensor.

A method for forming the receptor layer 26 is not particularly limited, and examples thereof include a method in which a composition including the gas-detecting compound is applied onto the gate insulating layer 24, and a drying treatment is carried out as necessary, thereby forming the receptor layer 26 and a method in which the gas-detecting compound is deposited on the gate insulating layer 24 by means of vapor deposition or sputtering, thereby forming the receptor layer 26.

[Substrate]

The substrate 20 is a base material supporting the respective members such as the gate electrode 22.

The type of the substrate 20 is not particularly limited, the substrate is mainly constituted of glass or a flexible resin sheet, and it is possible to use, for example, a plastic film. Examples of the plastic film include films made of polyethylene terephthalate (PET), polyethylene naphthalate (PEN), polyether ether ketone, polyphenylene sulfide, polyarylate, polyimide, polycarbonate (PC), cellulose triacetate (TAC), cellulose acetate propionate (CAP), or the like. As described above, when the plastic film is used, it is possible to reduce the weight, improve the portability, and improve the resistance to impacts more than in a case in which a glass substrate is used.

Meanwhile, in a case in which the gate electrode 22 described below also functions as the substrate, the substrate 20 may not be provided.

[Gate Electrode]

The gate electrode 22 is an electrode that is disposed on the substrate 20.

A material constituting the gate electrode 22 is not particularly limited as long as the material is conductive, and examples thereof include metal such as gold (Au), silver, aluminum (Al), copper, chromium, nickel, cobalt, titanium, platinum, magnesium, calcium, barium, and sodium; conductive oxides such as InO₂, SnO₂, and ITO; conductive high molecules such as polyaniline, polypyrrole, polythiophene, polyacetylene, and polydiacetylene; semiconductors such as silicon, germanium, potassium, and arsenic; carbon materials such as fullerene, carbon nanotubes, and graphite; and the like. Among these, metal is preferred, and silver or aluminum is more preferred.

The thickness of the gate electrode 22 is not particularly limited, but is preferably 20 to 1,000 nm.

The pattern shape of the gate electrode 22 is not particularly limited, and the optimal shape is appropriately selected.

A method for forming the gate electrode 22 is not particularly limited, and examples thereof include a method in which an etching treatment is carried out using well-known photolithography on a conductive thin film which is formed on the substrate 20 using a method such as vapor deposition or sputtering, thereby forming the gate electrode 22 and a method in which a mask having a predetermined pattern is disposed on the substrate 20, and vapor deposition, sputtering, or the like is carried out thereon, thereby forming the gate electrode 22.

In addition, the gate electrode 22 may be formed by directly carrying out patterning on the substrate 20 using a solution of a conductive high molecule or a dispersion liquid and an ink jet method or the gate electrode 22 may be formed from a coated film using photolithography or a laser abrasion method. Furthermore, it is also possible to use a method in which patterning is carried out using ink, conductive paste, or the like which includes a conductive high molecule or conductive fine particles and a printing method such as relief printing, intaglio printing, planography, or screen printing.

[Gate Insulating Layer]

The gate insulating layer 24 is a layer disposed on the substrate 20 so as to cover he gate electrode 22.

Examples of the material of the gate insulating layer 24 include polymers such as polymethyl methacrylate, polystyrene, polyvinyl phenol, polyimide, polycarbonate, polyester, polyvinyl alcohol, polyvinyl acetate, polyurethane, polysulfone, polybenzo-oxazole, polysilsesquioxane, epoxy resins, and phenolic resins; oxides such as silicon monoxide, silicon dioxide, aluminum oxide, and titanium oxide; nitrides such as silicon nitride; and the like. Among these materials, as the material of the gate insulating layer 24, an organic insulating material is preferably used from the viewpoint of handling properties.

In a case in which a polymer is used as the material of the gate insulating layer 24, it is preferably to jointly use a crosslinking agent (for example, melamine). When a crosslinking agent is jointly used, the polymer is crosslinked, and the durability of the gate insulating layer 24 being formed improves.

The thickness of the gate insulating layer 24 is not particularly limited, but is preferably 50 nm to 3 μm and more preferably 200 nm to 1 μm.

A method for forming the gate insulating layer 24 is not particularly limited, and examples thereof include a method in which a composition for forming the gate insulating layer including an organic insulating material is applied onto the substrate 20 on which the gate electrode 22 is formed, thereby forming the gate insulating layer 24, a method in which the gate insulating layer 24 is formed by means of vapor deposition or sputtering, and the like.

Meanwhile, the composition for forming the gate insulating layer may include a solvent (water or an organic solvent) as necessary. In addition, the composition for forming the gate insulating layer may include a crosslinking component. For example, when a crosslinking component such as melamine is added to an organic insulating material containing a hydroxyl group, it is also possible to introduce a crosslinking structure into the gate insulating layer 24.

A method for applying the composition for forming the gate insulating layer is not particularly limited, but wet processes such as application methods such as a spray coating method, a spin coating method, a blade coating method, a dip coating method, a casting method, a roll coating method, a bar coating method, and a die coating method and patterning methods such as ink jet are preferred.

In a case in which the gate insulating layer 24 is formed by applying the composition for forming the gate insulating layer, the gate insulating layer may be heated (baked) after the coating for the purpose of solvent removal and crosslinking.

[Organic semiconductor layer]

The organic semiconductor layer 28 is a layer disposed on the receptor layer 26 and is a layer that changes the electrical characteristics (particularly, electrical resistance) of the receptor layer when the adsorption of gas molecules occurs in the receptor layer 26.

The type of an organic semiconductor compound included in the organic semiconductor layer 28 is not particularly limited, and well-known organic semiconductor compounds can be used. Specific examples thereof include pentanes such as 6,13-bis(triisopropylsilylethynyl) pentacene (TIPS pentacene), tetramethylpentacene, and perfluoropentacene, anthradithiophenes such as TES-ADT (5,11-bis(triethylsilylethynyl) anthradithiophene), and diF-TES-ADT (2,8-difluoro-5,11-bis(triethylsilylethinyl) anthradithiophene), benzothienobenzothiophenes such as DPh-BTBT (2,7-diphenyl[1]benzothieno[3,2-b][1]benzothiophene) and Cn-BTBT (benzothienobenzothiophene), dinaphthothienothiophenes such as Cn-DNTT (dinaphtho[2,3-b:2′,3′-f]thieno[3,2-b]thiophene), dioxaanthanthrenes such as peri-Xanthenoxanthene, rubrenes, fullerenes such as C60, PCBM ([6,6]-Phenyl-C61-Butyric Acid Methyl Ester), phthalocyanines such as copper phthalocyanine and fluorinated copper phthalocyanine, polythiophenes such as P3RT (poly(3-alkylthiophene)), PQT (poly[5,5 ′-bis(3-dodecyl-2-thienyl1)-2,2′-bithiophene]), P3HT (poly(3-hexylthiophene)), and polythienothiophenes such as poly[2,5-bis(3-dodecylthiophene-2-yl)thieno[3,2-b]thiophene] (PBTTT).

Meanwhile, the organic semiconductor layer 28 may include a high-molecular-weight compound. The type of the high-molecular-weight compound is not particularly limited, and examples thereof include well-known high-molecular-weight compounds. A suitable aspect of the high-molecular-weight compound is a high-molecular-weight compound having a benzene ring (a high molecule having a repeating unit having a benzene ring group).

Examples of the high-molecular-weight compound include polystyrene, poly(α-methylstyrene), polyvinyl cinnamate, poly(4-vinylphenyl), poly(4-methylstyrene), and the like.

The thickness of the organic semiconductor layer 28 is not particularly limited, but is preferably 200 nm or less and more preferably 50 nm or less. The lower limit is not particularly limited, but is 10 nm or more in many cases.

A method for forming the organic semiconductor layer 28 is not particularly limited, and examples thereof include a method in which an organic semiconductor compound is deposited on the receptor layer 26 by means of vapor deposition or sputtering, thereby forming the organic semiconductor layer 28 (a dry method), a method in which an organic semiconductor composition including an organic semiconductor compound is applied onto the receptor layer 26, and a drying treatment is carried out as necessary, thereby forming the organic semiconductor layer 28 (a wet method), and the like. As described below, a method for forming the organic semiconductor layer 28 constituted of a polycrystal is preferably the above-described dry method.

A suitable aspect of the organic semiconductor layer 28 is preferably a polycrystalline layer (a layer constituted of a polycrystal or a layer having a polycrystalline structure). Meanwhile, the polycrystal refers to a crystal made up of a plurality of single crystals, and a plurality of these crystal grains may or may not be aligned to each other. In addition, the crystal grain refers to a fine single crystal which may partially include amorphous portions.

In a case in which the organic semiconductor layer 28 is a polycrystalline layer, when gas molecules permeate into the organic semiconductor layer 28 from the surface of the organic semiconductor layer 28 on a side opposite to the substrate 20 side, the gas molecules easily permeate into the layer through crystal grains, and consequently, the gas molecules easily reach the receptor layer 26.

The average particle diameter (average diameter) of crystal grains constituting the polycrystal is not particularly limited, but is 100 to 2,500 nm in many cases, and is preferably 100 to 1,000 nm and more preferably 100 to 600 nm since the detection sensitivity of the gas sensor is superior.

Regarding the method for measuring the average particle diameter of the crystal grains, the surface of the organic semiconductor layer 28 is observed using a microscope (for example, an atomic force microscope), the circle-equivalent diameters of at least 20 crystal particles are measured, and an arithmetic average value thereof is obtained. The circle-equivalent diameter refers to the diameter of a circle having the same area as the area of the two-dimensional image of an observed crystal particle.

[Source Electrode and Drain Electrode]

The source electrode 30 and the drain electrode 32 are electrodes disposed on the organic semiconductor layer 28 and are disposed to be spaced each other.

The source electrode 30 and the drain electrode 32 are rectangular electrodes that extend in a direction perpendicular to the direction in which both electrodes face each other.

Examples of a material constituting the source electrode 30 and the drain electrode 32 include the above-described materials constituting the gate electrode 22. In addition, examples of a method for forming the source electrode 30 and the drain electrode 32 include the above-described methods for forming the gate electrode 22.

The thickness of the source electrode 30 and the drain electrode 32 is not particularly limited, but is preferably 20 to 1,000 nm.

The channel length of the source electrode 30 and the drain electrode 32 is not particularly limited, but is preferably 5 to 30 μm.

The channel width of the source electrode 30 and the drain electrode 32 is not particularly limited, but is preferably 10 to 200 μm.

[Other Layers]

The organic transistor 10 may include layers other than the above-described members. For example, a self assembly mono layer may be disposed between the gate insulating layer 24 and the receptor layer 26. When a self assembly mono layer is disposed, the performance of the organic transistor 10 further improves, and the detection sensitivity further improves.

The type of a compound used to form the self assembly mono layer (SAM) is not particularly limited, but an organic compound which has a reactive functional group at one end of the molecule and has a substituent having a function of decreasing the surface energy at the other end is suitably used.

Examples of the compound used to form SAM include perfluorodecyltrichlorosilane [FDTS, (CF₃(CF₂)₇(CH₂)₂SiCl₃)], hexamethyldisilazane [HMDS, [(CH₃)₃Si]₂NH], octadecyltrichlorosilane [OTS, CH₃(CH₂)₁₇SiCl₃], hepta-decafluoro-1,1,2,2-tetrahydrodecyltrichlorosilane [FDTS, CF₃(CF₂)₅(CH₂)₂SiCl₃], tridecafluoro-1,1,2,2-tetrahydrooctyltrichlorosilane [FOTS, CF₃(CF₂)₅(CH₂)₂SiCl₃], tridecafluoro-1,1,2,2-tetrahydrooctyltriethoxysilane [FOTES, CF₃(CF₂)₅(CH₂)₂Si(OC₂H₅)₃], tridecafluoro-1,1,2,2-tetrahydrooctylmethyldichlorosilane [FOMDS, CF₃(CF₂)₅(CH₂)₂Si(CH₃)Cl₂], tridecafluoro-1,1,2,2-tetrahydrooctyldimethylchlorosilane [FOMMS, CF₃(CF₂)₅(CH₂)₂Si(CH₃)₂Cl), dimethyldichlorosilane [DDMS, (CH₃)₂SiCl₂], and the like.

The thickness of the self assembly mono layer is not particularly limited, but is the thickness of one molecule of a compound used to form SAM in many cases, and is 1 to 3 nm in many cases.

A method for forming the self assembly mono layer is not particularly limited, and examples thereof include a method in which a composition including a compound used to form SAM is applied onto the gate insulating layer 24 and a washing treatment is carried out as necessary.

In addition, a carrier injection layer may be disposed between the organic semiconductor layer 28 and the source electrode 30 (or the drain electrode 32). The carrier injection layer functions as a layer that forms charge migration between an organic semiconductor and the carrier injection layer and reduce the contact resistance so that carriers are effectively injected into the organic semiconductor from an electrode even at a low voltage.

The carrier injection layer is formed using, for example, tetrafluorotetracyanodimethane (F4-TCNQ), hexaazatriphenylenehexacarbonitrile (HAT-CN), molybdenum oxide (MoOx), or the like.

[Measurement Portion]

The measurement portion is a portion (device) which is connected to the organic transistor, detects the electrical characteristic change of the organic transistor, and measures (computes) gas concentrations.

The type of the changes in the electrical characteristics of the organic transistor 10 which is detected in the measurement portion is not particularly limited, and examples thereof include the changes in the current values between the source electrode and the drain electrode (the current values of drain currents), the changes in carrier mobility, voltage changes, and the like. Among these, it is preferable to detect the changes in the current values between the source electrode and the drain electrode (the current values of drain currents) from the viewpoint of ease of measurement.

Regarding the constitution of the measurement portion, for example, in a case in which the changes in the current values of drain currents are measured, a detection portion including at least a power supply and an ammeter is included. Meanwhile, generally, the power supply is connected to the source electrode and the drain electrode in the organic transistor.

In addition, the measurement portion further includes a conversion portion that computes the concentrations of gas molecules which are a detection subject on the basis of the amounts of the detected changes in the electrical characteristics of the organic transistor (for example, the amounts of changes in the current values of drain currents). Meanwhile, the concentrations of gas molecules can be computed from previously-produced calibration curves in which the relationship between the amount of changes in the electrical characteristics and the concentration of gas molecules is specified.

[Detection Subject]

In the gas sensor having the above-described constitution, a variety of gas molecules (for example, acetone, ethanol, and toluene) can be detected depending on the gas-detecting compound being used. Among these, the detection subject is preferably gas molecules in the exhaled air of human beings (predetermined gas molecules), and more specific examples thereof include acetone, ethanol, and the like.

### Second Embodiment

Hereinafter, a second embodiment of the gas sensor of the present invention will be described with reference to the drawing. FIG. 2 is a cross-sectional view of the organic transistor included in the gas sensor of the present invention.

The organic transistor 110 used in the second embodiment of the gas sensor includes the substrate 20, the gate electrode 22 disposed on the substrate 20, the gate insulating layer 24 disposed so as to cover the gate electrode 22, the source electrode 30 and the drain electrode 32 disposed to be spaced each other on the gate insulating layer 24, the receptor layer 26 that covers the surface of the gate insulating layer 24 between the source electrode 30 and the drain electrode 32, and the organic semiconductor layer 28 which is disposed on the receptor layer 26 so as to cover the receptor layer 26 and is connected to the source electrode 30 and the drain electrode 32.

The second embodiment of the gas sensor has the same constitution as that of the first embodiment of the gas sensor except for the fact that the locations of the layers in the organic transistor being used are different, and thus the same constituent element will be given the same reference sign and will not be described.

Hereinafter, the order of the respective layers in the organic transistor 110 will be described in detail.

The organic transistor 110 is a so-called bottom-gate/top-contact type organic transistor, and the source electrode 30 and the drain electrode 32 are disposed on the gate insulating layer 24.

Predetermined gas molecules which are a detection subject reach the receptor layer 26 through the organic semiconductor layer 28 and are adsorbed. When the gas molecules are adsorbed in the receptor layer 26, the electrical resistance changes in the organic semiconductor layer 28 disposed adjacent to the receptor layer 26, and consequently, the electrical characteristics of the transistor change.

In FIG. 2, the receptor layer 26 is disposed only on the gate insulating layer 24 between the source electrode 30 and the drain electrode 32, but the constitution is not limited to this aspect, and the receptor layer may be disposed on the entire surface of the gate insulating layer 24. That is, the receptor layer may be disposed on the entire surface of the gate insulating layer 24, and the source electrode 30 and the drain electrode 32 are disposed on the receptor layer. Therefore, the receptor layer 26 needs to be disposed at least between the gate insulating layer 24 and the organic semiconductor layer 28.

### EXAMPLES

Hereinafter, examples will be described, but the present invention is not limited thereto.

### Example 1

An aluminum (Al) electrode was formed on a predetermined location on a washed glass substrate in a thickness of 30 nm using a vacuum vapor deposition method, thereby producing a gate electrode. Next, a propylene glycol-1-methyl ether acetate (PGMEA) solution including polyvinyl alcohol (PVA) (the content of PVA was 10% by mass of the total mass of the solution) and a PGMEA solution including melamine (the content of melamine was 10% by mass of the total mass of the solution) were mixed together in a mass ratio of 1:1, the obtained solution was applied onto the gate electrode using a spin coating method so as to form a film, and then an annealing treatment was carried out for one hour at 150° C. on a hot plate, thereby forming a gate insulating layer (thickness: 230 nm). After that, a toluene solution of tetraphenylporphyrin was applied onto the gate insulating layer and was dried by means of vacuum heating, thereby forming a receptor layer (thickness: 10 nm). 2,7-Dioctyl[1]benzothieno[3,2-b][1]benzothiophene (C8-BTBT) was deposited on the obtained receptor layer, thereby forming an organic semiconductor layer (thickness: 50 nm). Next, tetrafluorotetracyanodimethane (F4-TCNQ) was deposited in a predetermined location on the organic semiconductor layer using a metal mask so as to form a carrier injection layer (thickness: 4 nm), and furthermore, gold was deposited on the carrier injection layer so as to form a source electrode (thickness: 50 nm) and a drain electrode (thickness: 50 nm), thereby producing an organic transistor. The obtained organic transistor had the same constitution as in FIG. 1.

In addition, the organic semiconductor layer was a polycrystalline layer, and the average particle diameter of crystal grains constituting the polycrystal was 350 nm. Meanwhile, in the examples, the average particle diameter was obtained by observing the surface of the organic semiconductor layer using an atomic force microscope (manufactured by Hitach High-Tech Science Corporation), measuring the circle-equivalent diameters of 20 crystal particles, and arithmetically averaging the values.

In a sealed chamber, the source electrode and the drain electrode in the obtained organic transistor were connected to a prober (measurement portion), and the measurement of transistor characteristics (drain current values) began in a dried nitrogen atmosphere. Gas obtained by mixing dried nitrogen and acetone at an arbitrary ratio was caused to pass through the chamber, and changes in the transistor characteristics (current changes) were measured before and after the passing of the gas. As a result, 100 ppm of acetone could be detected.

### Example 2

An organic transistor was produced according to the same order as in Example 1 except for the fact that a tetraphenylporphyrin-manganese complex was used instead of tetraphenylporphyrin. After that, the detection of acetone was carried out in the same order as in Example 1 using the obtained organic transistor, and consequently, 100 ppm of acetone could be detected.

Meanwhile, the organic semiconductor layer was a polycrystalline layer, and the average particle diameter of the crystal grains constituting the polycrystal was 450 nm.

### Example 3

An organic transistor was produced according to the same order as in Example 1 except for the fact that phthalocyanine was used instead of tetraphenylporphyrin. After that, the detection of acetone was carried out in the same order as in Example 1 using the obtained organic transistor, and consequently, 100 ppm of acetone could be detected.

Meanwhile, the organic semiconductor layer was a polycrystalline layer, and the average particle diameter of the crystal grains constituting the polycrystal was 350 nm.

### Example 4

The detection of ethanol was carried out in the same order as in Example 1 using the organic transistor obtained in Example 1 except for the fact that ethanol was used as a detection subject instead of acetone, and consequently, 100 ppm of ethanol could be detected.

Meanwhile, the organic semiconductor layer was a polycrystalline layer, and the average particle diameter of the crystal grains constituting the polycrystal was 600 nm.

### Example 5

An organic transistor was produced according to the same order as in Example 1 except for the fact that an organic semiconductor layer was formed in a drop cast using a solution obtained by dissolving C8-BTBT in toluene (the concentration of C8-BTBT: 1% by mass). After that, the detection of acetone was carried out in the same order as in Example 1 using the obtained organic transistor, and consequently, it was not possible to detect 100 ppm of acetone, but 500 ppm of acetone could be detected.

Meanwhile, the organic semiconductor layer was a polycrystalline layer, and the average particle diameter of the crystal grains constituting the polycrystal was 2 μm.

### Comparative Example 1

An organic transistor was produced according to the same order as in Example 1 except for the fact that the receptor layer was not formed. The organic semiconductor layer was a polycrystalline layer, and the average particle diameter of the crystal grains constituting the polycrystal was 350 nm. After that, the detection of acetone was carried out in the same order as in Example 1 using the obtained organic transistor, and consequently, no changes in the transistor characteristics of the organic transistor were observed even at an acetone concentration of 500 ppm, and it was not possible to detect acetone.

### Comparative Example 2

An Al electrode was formed on a predetermined location on a washed glass substrate in a thickness of 30 nm using a vacuum vapor deposition method, thereby producing a gate electrode. Next, a propylene glycol-1-methyl ether acetate (PGMEA) solution including polyvinyl alcohol (PVA) (the content of PVA was 10 parts by mass of the total mass of the solution) and a PGMEA solution including melamine (the content of melamine was 10% by mass of the total mass of the solution) were mixed together in a mass ratio of 1:1, the obtained solution was applied onto the gate electrode using a spin coating method so as to form a film, and then an annealing treatment was carried out for one hour at 150° C. on a hot plate, thereby forming a gate insulating layer (thickness: 230 nm). After that, 2,7-dioctyl[1]benzothieno[3,2-b][1]benzothiophene (C8-BTBT) was deposited on the gate insulating layer, thereby forming an organic semiconductor layer (thickness: 50 nm). Next, tetrafluorotetracyanodimethane (F4-TCNQ) was deposited in a predetermined location on the organic semiconductor layer using a metal mask so as to faun a carrier injection layer (thickness: 4 nm), furthermore, gold was deposited on the carrier injection layer so as to foi in a source electrode (thickness: 50 nm) and a drain electrode (thickness: 50 nm), and then, a toluene solution of tetraphenylporphyrin was applied onto the organic semiconductor layer and was dried by means of vacuum heating so as to form a receptor layer (thickness: 10 nm), thereby producing an organic transistor. The obtained organic transistor had the same constitution as in FIG. 1. The organic semiconductor layer was a polycrystalline layer, and the average particle diameter of the crystal grains constituting the polycrystal was 550 nm.

The detection of acetone was carried out in the same order as in Example 1 using the obtained organic transistor, and consequently, no changes in the transistor characteristics of the organic transistor were observed even at an acetone concentration of 500 ppm, and it was not possible to detect acetone.

The results of Examples 1 to 5 and Comparative Examples 1 and 2 described above are summarized in Table 1.

In Table 1, in the column of “location of receptor layer”, “A” indicates that the receptor layer is located between the gate insulating layer and the organic semiconductor layer, and “B” indicates that the receptor layer is provided at a location different from that of the above-described A aspect.

In Table 1, in the column of “result”, “A” indicates a case in which gas molecules are detected at a concentration of 100 ppm, “B” indicates a case in which gas molecules cannot be detected at a concentration of 100 ppm, but can be detected at 500 ppm, and “C” indicates a case in which gas molecules cannot be detected even at a concentration of 500 ppm.

TABLE 1

| | Presence | | | Average | | |
|---|---|---|---|---|---|---|
| | or | Location | | particle | | |
| | absence of | of | | diameter | | |
| | receptor | receptor | Compound in | of crystal | Measurement | |
| | layer | layer | receptor layer | grains (nm) | subject | Result |
| Example 1 | Presence | A | Tetraphenylporphyrin | 350 | Acetone | A |
| Example 2 | Presence | A | Tetraphenylporphyrin- | 450 | Acetone | A |
| | | | manganese complex | | | |
| Example 3 | Presence | A | Phthalocyanine | 350 | Acetone | A |
| Example 4 | Presence | A | Tetraphenylporphyrin | 600 | Ethanol | A |
| Example 5 | Presence | A | Tetraphenylporphyrin | 2000 | Acetone | B |
| Comparative | Absence | — | — | 350 | Acetone | C |
| Example 1 | | | | | | |
| Comparative | Presence | B | Tetraphenylporphyrin | 550 | Acetone | C |
| Example 2 | | | | | | |

As shown in Table 1, according to the gas sensor of the present invention, gas molecules which were a detection subject could be detected with high detection sensitivity.

Particularly, it was confirmed that, in a case in which the organic semiconductor layer is a polycrystalline layer and the average particle diameter of crystal grains in the polycrystal is 1 μm or less, the effect is superior.

On the other hand, in Comparative Examples 1 and 2 in which the predetermined constitution was not provided, the desired effect could not be obtained. Particularly, in Comparative Example 2 in which the constitution as described in JP2006-258661A was provided, the desired effect could not be obtained.

### EXPLANATION OF REFERENCES

10, 110: organic transistor

20: substrate

22: gate electrode

24: gate insulating layer

26: receptor layer

28: organic semiconductor layer

30: source electrode

What is claimed is:

1. A gas sensor comprising:
a bottom-gate type organic transistor including a source electrode, a drain electrode, a gate electrode, a gate insulating layer, an organic semiconductor layer, and a receptor layer which is disposed between the gate insulating layer and the organic semiconductor layer and includes a compound that interacts with gas molecules which are a detection subject.

2. The gas sensor according to claim 1,
wherein the organic transistor includes the gate electrode, the gate insulating layer disposed on the gate electrode, the receptor layer disposed on the gate insulating layer, the organic semiconductor layer disposed on the receptor layer,and the source electrode and the drain electrode disposed on the organic semiconductor layer.

3. The gas sensor according to claim 1,
wherein the organic transistor includes the gate electrode, the gate insulating layer disposed on the gate electrode, the source electrode and the drain electrode disposed on the gate insulating layer, the receptor layer that covers a surface of the gate insulating layer between the source electrode and the drain electrode, and the organic semiconductor layer disposed on the receptor layer.

4. The gas sensor according to claim 1,
wherein the organic semiconductor layer is a polycrystalline layer.

5. The gas sensor according to claim 1,
wherein the compound that interacts with gas molecules which are a detection subject has an amino group.

6. The gas sensor according to claim 1,
wherein a thickness of the receptor layer is 10 to 50 nm.

7. The gas sensor according to claim 1,
wherein a thickness of the organic semiconductor layer is 50 nm or less.

8. The gas sensor according to claim 1,
wherein the gas molecules which are a detection subject are gas molecules included in exhaled air of human beings.

9. The gas sensor according to claim 1,
wherein the gas molecules which are a detection subject are acetone.

10. The gas sensor according to claim 1,
wherein the gas molecules which are a detection subject are ethanol.

11. A bottom-gate type organic transistor for a gas sensor, comprising:
a source electrode, a drain electrode, a gate electrode, a gate insulating layer, an organic semiconductor layer, and a receptor layer which is disposed between the gate insulating layer and the organic semiconductor layer and includes a compound that interacts with gas molecules which are a detection subject.

12. The gas sensor according to claim 2,
wherein the organic semiconductor layer is a polycrystalline layer.

13. The gas sensor according to claim 3,
wherein the organic semiconductor layer is a polycrystalline layer.

14. The gas sensor according to claim 2,
wherein the compound that interacts with gas molecules which are a detection subject has an amino group.

15. The gas sensor according to claim 3,
wherein the compound that interacts with gas molecules which are a detection subject has an amino group.

16. The gas sensor according to claim 4,
wherein the compound that interacts with gas molecules which are a detection subject has an amino group.

17. The gas sensor according to claim 2,
wherein a thickness of the receptor layer is 10 to 50 nm.

18. The gas sensor according to claim 3,
wherein a thickness of the receptor layer is 10 to 50 nm.

19. The gas sensor according to claim 4,
wherein a thickness of the receptor layer is 10 to 50 nm.

20. The gas sensor according to claim 5,
wherein a thickness of the receptor layer is 10 to 50 nm.
